(19) United States
(12) Patent Application Publication
(10) Pub. No.: US 2025/0025044 A1
(43) Pub. Date: Jan. 23, 2025

(54) ANGIO-OCT-BASED DYNAMIC FUNCTIONAL RETINAL BLOOD FLOW IMAGING APPARATUS AND IMAGING METHOD THEREOF

(71) Applicant: THE EYE HOSPITAL OF WENZHOU MEDICAL UNIVERSITY, Wenzhou (CN)

(72) Inventors: Shaodan Zhang, Wenzhou (CN); Meixiao Shen, Wenzhou (CN); Yuanbo Liang, Wenzhou (CN); Dexi Zhu, Wenzhou (CN); Ruirong You, Wenzhou (CN); Sigeng Lin, Wenzhou (CN)

(21) Appl. No.: 18/709,527

(22) PCT Filed: Sep. 21, 2022
(86) PCT No.: PCT/CN2022/120223

§ 371 Date: May 13, 2024

(30) Foreign Application Priority Data
Jun. 27, 2022 (CN) 202210738150.7

(51) Int. Cl. A61B 3/12; A61B 3/00; A61B 3/10; A61B 3/14

(52) U.S. Cl. CPC A61B 3/1241; A61B 3/0025; A61B 3/102; A61B 3/14

(57) ABSTRACT

Provided is an angio-OCT-based dynamic functional retinal blood flow imaging apparatus, comprising an angio-OCT sample optical path and an angio-OCT signal acquisition path, and further comprising a visual stimulation module arranged on the same axis as a sample arm optical path, the visual stimulation module projecting a stimulation image to different parts of the retina in the eye by means of a hot mirror DM. Further provided is an imaging method for an angio-OCT visual stimulation-induced retinal dynamic functional blood flow change image of an angio-OCT-based dynamic functional retinal blood flow imaging apparatus. The apparatus and the method focus on the functional characteristics of a visual system, and based on the special functional unit, “vascular nerve coupling”, of a central nervous system, dynamic functional retinal blood flow imaging combined with visual stimulation is developed on the basis of existing angio-OCT imaging technology.

### TECHNICAL FIELD

The present invention relates to the technical field of OCT, and more particularly, to an angio-OCT-based dynamic functional retinal blood flow imaging apparatus and an imaging method thereof.

### BACKGROUND

A basic imaging principle of a current angio-OCT imaging technology is to separate a stable signal generated by a static tissue and an irregular signal generated by moving particles (red blood cells) from light signals backscattered by a sample. Imaging signals of two scans were subtracted to filter out a static signal, so as to display information of moving particles and reflect the distribution, morphology and density of small retinal blood vessels, which provides a powerful auxiliary detection and evaluation method for judgment of clinical diagnosis and treatment effects of retinal vascular diseases. (1) Many systemic and retinal vascular diseases are first manifested as dysfunction (functional abnormality) of vascular regulation before a structural change of blood vessels, while the distribution, density, blood flow in a resting state, etc. of the blood vessels have not been significantly changed. For example, it has been found under research that the hemodynamics of the central retinal artery and the ophthalmic artery in diabetic patients before the onset of diabetic retinopathy are significantly abnormal, which is manifested in the decrease of systolic peak blood flow velocity (PSV), end-diastolic blood flow velocity (EDV), and temporal mean blood flow velocity (TAV) compared with a healthy control group, while a resistance index (RI) of the blood vessels is significantly increased. At present, the angio-OCT imaging technology does not have a capability to detect these conditions. (2) On the other hand, the brain and retina have a functional unit of “vascular nerve coupling”, and activities of neurons are accompanied by an increase in local blood flow. Our previous studies on the visual cortex of the brain using functional magnetic resonance imaging and ultrasound Doppler technologies, as well as relevant foreign studies, have confirmed that before structural changes of central neurons, abnormal neural activities may cause changes in blood perfusion in their areas. The existence of “vascular nerve coupling” provides a new idea for the early diagnosis and disease progression judgment of nerve-related diseases in the central nervous system using changes in blood flow. It is also the theoretical basis of the technical design of this patent. The existing angio-OCT technology does not have a stimulation element that induces retinal neuron activities.

In addition to the OCT technology, other methods that may be used to measure retinal blood flow include an ultrasound Doppler technology, a laser blood flow imaging technology, blood oxygen saturation detection, etc. Due to resolution and other reasons, these methods do not have the capability to detect hemodynamic changes in the retinal microcirculation, and thus do not have the capability to meet the above two detection requirements.

### SUMMARY OF THE INVENTION

To solve the technical defects existing in the prior art, the present invention provides an angio-OCT-based dynamic functional retinal blood flow imaging apparatus and an imaging method thereof. Through the dynamic functional detection of retinal blood vessels, early diagnosis may be performed for retinal vascular diseases and indirectly for retinal neurological diseases, assisted with disease progression and prognosis.

A technical solution adopted by the present invention is as follows: an angio-OCT-based dynamic functional retinal blood flow imaging apparatus includes an angio-OCT sample optical path and an angio-OCT signal acquisition path, and further includes a visual stimulation module arranged on the same axis as a sample arm optical path, the visual stimulation module projecting a stimulation image to different parts of the retina in the eye by means of a hot mirror DM.

The imaging apparatus further includes a data acquisition and control module, the data acquisition and control module controlling an acquisition trigger signal for the angio-OCT signal acquisition path.

The visual stimulation module includes a generator and an optotype display, the optotype display displaying that the stimulation image is projected to the retina in the eye by means of the hot mirror DM, and the generator being controlled by the data acquisition and control module to generate a control signal for the stimulation image of the optotype display.

The optotype display of the visual stimulation module includes a resting mode and a stimulation mode; in the resting mode, black screens of the same size including a central fixation optotype are used as a baseline image; in the stimulation mode, a black and white flipping checkerboard graph under a black background is used as the stimulation image; and a flipping frequency of the flipping checkerboard graph in the stimulation mode is synchronously controlled by a trigger signal for the data acquisition and control module to control the generator and the acquisition trigger signal for the angio-OCT signal acquisition path.

The angio-OCT sample optical path includes a light source, a lens, an X/Y galvanometer, a lens, and an eyeball sequentially from the light source to the eyeball; the light source is connected to the adjacent lens through an optical fiber; and an optical fiber coupling is also provided on the optical fiber.

The hot mirror DM is arranged between the X/Y galvanometer and the eyeball, the hot mirror DM projecting the stimulation image of the visual stimulation module to different parts of the retina in the eye.

The angio-OCT signal acquisition path includes an optical fiber coupling, a polarization controller and lens, an optical fiber coupling, a balance detector BD, and the data acquisition and control module sequentially from the optical fiber coupling to the data acquisition and control module; and components of the angio-OCT signal acquisition path are all connected by the optical fiber.

The data acquisition and control module is a data acquisition and control terminal DAQ.

An imaging method for an angio-OCT visual stimulation-induced retinal dynamic functional blood flow change image includes the following steps: successively acquiring, according to an acquisition trigger signal controlled by the data acquisition and control module, retinal angio-OCT vascular signals of the same eye under a black screen condition in a resting mode of a visual stimulation module and under a checkerboard stimulation condition of a stimulation mode; analyzing a difference between vascular signals under the two states by an existing angio-OCT algorithm in a system; quantitatively analyzing a blood flow density in an area having the difference; and annotating areas of blood flow increase, blood flow decrease and no change after a visual stimulation by a heat map to obtain the visual stimulation-induced retinal dynamic functional blood flow change image.

s under the two states by an existing angio-OCT algorithm in a system; quantitatively analyzing a blood flow density in an area having the difference; and annotating areas of blood flow increase, blood flow decrease and no change after a visual stimulation by a heat map to obtain the visual stimulation-induced retinal dynamic functional blood flow change image.

The present invention has the following beneficial effects: the present invention provides an angio-OCT-based dynamic functional retinal blood flow imaging apparatus and an imaging method thereof, which focus on the functional characteristics of a visual system, and based on the special functional unit, “vascular nerve coupling”, of a central nervous system, dynamic functional retinal blood flow imaging combined with visual stimulation is developed on the basis of existing angio-OCT imaging technology, thereby further improving the ability of existing angio-OCT in the aspects of early diagnosis and disease progression judgment of retinal vascular diseases, and developing the detection capability of angio-OCT on early diagnosis and disease progression of retinal neurological diseases such as glaucoma.

### BRIEF DESCRIPTION OF THE DRAWINGS

FIG. 1 is a schematic diagram of an angio-OCT-based dynamic functional retinal blood flow imaging apparatus.

FIG. 2 shows a dynamic functional angio-OCT detection result diagram of a normal healthy eye, in which A is a peripapillary blood flow density of a static baseline when a checkerboard stimulation is not given; and B is a peripapillary blood flow density after the same eye accepts the checkerboard stimulation.

FIG. 3 is a schematic diagram of dynamic functional angio-OCT of a glaucoma patient, in which A shows a peripapillary blood flow density of the static baseline when the checkerboard stimulation is not given, in which decreased and absent local vascular density in the lower temporal area (a blue wedge-shaped area indicated by a red arrow) may be seen; and B shows a change in peripapillary blood flow density after the same eye accepts the checkerboard stimulation.

### DETAILED DESCRIPTION OF THE INVENTION

The technical solutions in the embodiments of the present invention will be described clearly and completely in conjunction with the accompanying drawings in the embodiments of the present invention. Apparently, the described embodiments are merely some embodiments, rather than all embodiments, of the present invention. Based on the embodiments of the present invention, all other embodiments derived by a person of ordinary skill in the art without creative efforts shall fall within the protection scope of the present invention.

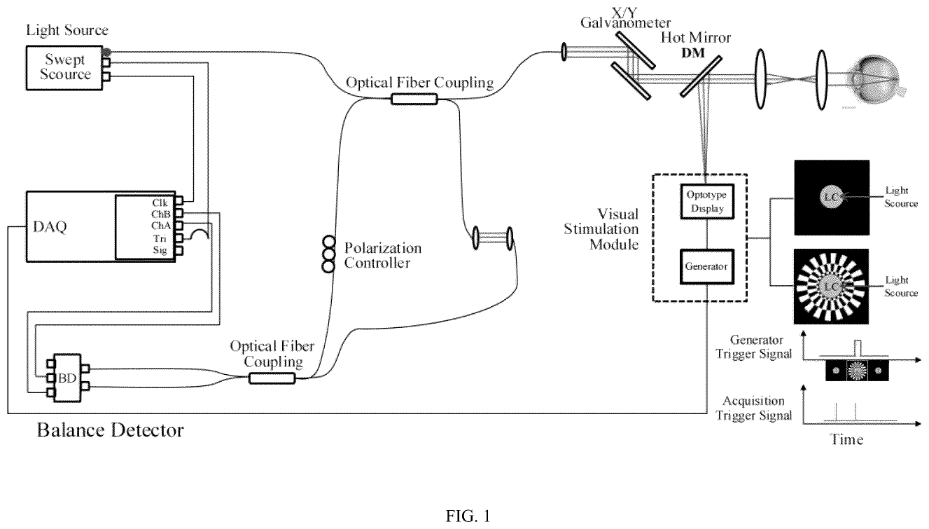

According to the present invention, on the basis of an angio-OCT system, a visual stimulation module coaxial with a sample arm optical path is added to an optical path structure of a device sample, which can realize a projection stimulation of a flipping checkerboard graph in different parts of the retina, and a flipping frequency may be adjusted (for example, for glaucoma vascular function detection, the frequency is set to 8 Hz) according to an application scenario. The system is shown in FIG. 1. The flipping frequency of the graphic stimulation is synchronously controlled by a trigger signal generated by a DAQ board card generator in the system and a signal acquisition trigger signal of Angio-OCT, and a signal control mode is shown in FIG. 1. Black screens of the same size are used as a baseline in a corresponding resting state in the visual stimulation module, and a flipping checkerboard graph is used in a stimulation condition (theoretically, this patent contains arbitrary stimulation graph). Retinal angio-OCT vascular signals of the same eye under a black screen condition and under a checkerboard stimulation condition are acquired successively. A difference between vascular signals under the two states is analyzed by an angio-OCT algorithm in the system. A blood flow density in an area having the difference is quantitatively analyzed. Areas of blood flow increase, blood flow decrease and no change after a visual stimulation by a heat map are annotated to obtain the visual stimulation-induced retinal dynamic functional blood flow change image.

The imaging apparatus further includes a data acquisition and control module, the data acquisition and control module controlling an acquisition trigger signal for the angio-OCT signal acquisition path.

The visual stimulation module includes a generator and an optotype display, the optotype display displaying that the stimulation image is projected to the retina in the eye by means of a hot mirror DM, and the generator being controlled by the data acquisition and control module to generate a control signal for a stimulation image of the optotype display.

The optotype display of the visual stimulation module includes a resting mode and a stimulation mode. In the resting mode, black screens of the same size are used as a baseline image. In the stimulation mode, a flipping checkerboard graph is used as the stimulation image. A flipping frequency of the flipping checkerboard graph in the stimulation mode is synchronously controlled by a trigger signal for the data acquisition and control module to control the generator and the acquisition trigger signal for the angio-OCT signal acquisition path.

The angio-OCT sample optical path includes a light source, a lens, an X/Y galvanometer, a lens, and an eyeball sequentially from the light source to the eyeball; the light source is connected to the adjacent lens through an optical fiber; and an optical fiber coupling is also provided on the optical fiber.

The hot mirror DM is arranged between the X/Y galvanometer and the eyeball, the hot mirror DM projecting the stimulation image of the visual stimulation module to different parts of the retina in the eye.

The angio-OCT signal acquisition path includes an optical fiber coupling, a polarization controller and lens, an optical fiber coupling, a balance detector BD, and the data acquisition and control module sequentially from the optical fiber coupling to the data acquisition and control module; and components of the angio-OCT signal acquisition path are all connected by the optical fiber.

### Effect Verification

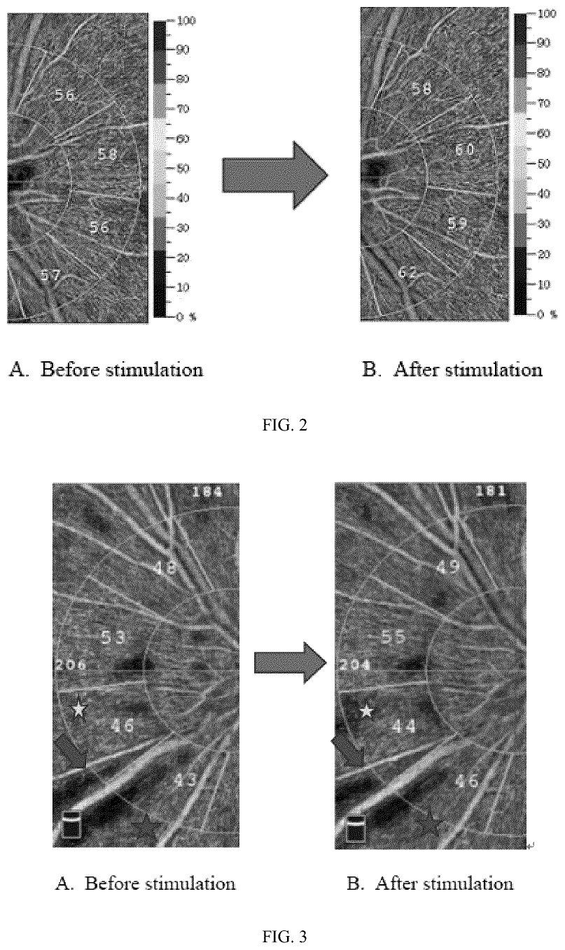

1. A normal healthy eye is verified, and a peripapillary blood flow density increases after a visual stimulation is accepted. As shown in FIG. 2, it can be seen that the vascular density in each area has increased compared with a baseline, indicating good neuron activities.

2. A glaucoma patient is verified; and after the visual stimulation is accepted, the vascular density in a defective area of a retinal nerve fiber layer decreases, while the vascular density in the adjacent normal area increases, and results are shown in FIG. 3. It can be seen that the blood flow density in the upper temporal area of an optic disk increases, while the blood flow density in the lower temporal part decreases (yellow asterisk), indicating that the neuron activities here are weakened and there may be disease progression. An increase in blood flow density (red asterisk) in some areas indicates passable neuron activities here.

Notes to technical personnel: although the present invention has been described in accordance with the above specific embodiments, the invention idea of the present invention is not limited to this invention, and any modification using the idea of the present invention will be included in the protection scope of this patent.

The above is only preferred embodiments of the present invention, the protection scope of the present invention is not limited to the above embodiments, all technical solutions belonging to the ideas of the present invention are within the protection scope of the present invention. It should be noted that those of ordinary skill in the art may also make several improvements and modifications without departing from the principles of the present invention, which should be considered as the protection scope of the present invention.

What is claimed is:

1. An angio-OCT-based dynamic functional retinal blood flow imaging apparatus, comprising an angio-OCT sample optical path and an angio-OCT signal acquisition path, and further comprising a visual stimulation module arranged on the same axis as the sample arm optical path, the visual stimulation module projecting a stimulation image to different parts of the retina in the eye by means of a hot mirror DM.

2. The angio-OCT-based dynamic functional retinal blood flow imaging apparatus according to claim 1, wherein the imaging apparatus further comprises a data acquisition and control module, the data acquisition and control module controlling an acquisition trigger signal for the angio-OCT signal acquisition path.

3. The angio-OCT-based dynamic functional retinal blood flow imaging apparatus according to claim 2, wherein the visual stimulation module comprises a generator and an optotype display, the optotype display displaying that the stimulation image being projected to the retina in the eye by means of the hot mirror DM, and the generator being controlled by the data acquisition and control module to generate a control signal for the stimulation image of the optotype display.

4. The angio-OCT-based dynamic functional retinal blood flow imaging apparatus according to claim 3, wherein the optotype display of the visual stimulation module comprises a resting mode and a stimulation mode; in the resting mode, black screens of the same size including a central fixation optotype are used as baseline images; in the stimulation mode, a black and white flipping checkerboard graph under a black background is used as the stimulation image; and a flipping frequency of the flipping checkerboard graph in the stimulation mode is synchronously controlled by a trigger signal for the data acquisition and control module to control the generator and the acquisition trigger signal for the angio-OCT signal acquisition path.

5. The angio-OCT-based dynamic functional retinal blood flow imaging apparatus according to claim 1, wherein the angio-OCT sample optical path comprises a light source, a lens, an X/Y galvanometer, a lens, and an eyeball sequentially from the light source to the eyeball; the light source is connected to the adjacent lens through an optical fiber; and an optical fiber coupling is also provided on the optical fiber.

6. The angio-OCT-based dynamic functional retinal blood flow imaging apparatus according to claim 5, wherein the hot mirror DM is arranged between the X/Y galvanometer and the eyeball, and the hot mirror DM projects the stimulation image of the visual stimulation module to different parts of the retina in the eye.

7. The angio-OCT-based dynamic functional retinal blood flow imaging apparatus according to claim 5, wherein the angio-OCT signal acquisition path comprises an optical fiber coupling, a polarization controller and lens, an optical fiber coupling, a balance detector BD, and the data acquisition and control module sequentially from the optical fiber coupling to the data acquisition and control module; and components of the angio-OCT signal acquisition path are all connected by the optical fiber.

8. The angio-OCT-based dynamic functional retinal blood flow imaging apparatus according to claim 1, wherein the data acquisition and control module is a data acquisition and control terminal DAQ.

9. An imaging method for an angio-OCT visual stimulation-induced retinal dynamic functional blood flow change image of the angio-OCT-based dynamic functional retinal blood flow imaging apparatus according to claim 1, comprising the following steps: successively acquiring, according to an acquisition trigger signal controlled by the data acquisition and control module, retinal angio-OCT vascular signals of the same eye under a black screen condition in a resting mode of a visual stimulation module and under a checkerboard stimulation condition of a stimulation mode; analyzing a difference between vascular signals under the two states by an existing angio-OCT algorithm in a system; quantitatively analyzing a blood flow density in an area having the difference; and annotating areas of blood flow increase, blood flow decrease and no change after visual stimulation by a heat map to obtain the visual stimulation-induced retinal dynamic functional blood flow change image.
